(19) United States
(12) Patent Application Publication
(10) Pub. No.: US 2019/0148020 A1
(43) Pub. Date: May 16, 2019

(54) INTEGRATED PLATFORM FOR CONNECTING PHYSIOLOGICAL PARAMETERS DERIVED FROM DIGITAL HEALTH DATA TO MODELS OF MORTALITY, MORBIDITY, LIFE EXPECTANCY AND LIFESTYLE INTERVENTIONS

(71) Applicant: LifeQ Global Limited, San Gwann, MT (US)

(72) Inventor: Franco Bauer DU PREEZ, Cumming, GA (US)

(21) Appl. No.: 16/190,080

(22) Filed: Nov. 13, 2018

Related U.S. Application Data
(60) Provisional application No. 62/585,373, filed on Nov. 13, 2017.

(51) Int. Cl. G16H 50/30; G16H 50/20; A61B 5/024; G16H 50/50

(52) U.S. Cl. CPC G16H 50/30; G16H 50/20; G16H 15/00; G16H 50/50; A61B 5/02405

(57) ABSTRACT

Embodiments include methods by which mathematical models are applied to data from at least two interrelated physiological parameters employed in parallel with data gathered from various sources to provide an end user (e.g., individual or group of individuals) or consented third party (e.g., insurer of the user) with an estimate of how the physiological parameters will change in the future and a number of years indicative of the terminal age and/or health and life expectancy of the user. Furthermore, utilizing the translation from a lifestyle intervention (e.g., cessation of smoking or commencement of an exercise program) to the physiological parameters from historical data (e.g., wearable data) may permit display of a lifestyle intervention pattern and a current and planned physiological trajectory. The output may be quantified translation from lifestyle intervention to real time health and life expectancy and may be calculated from past and present data and hypothetical planned interventions.

### CROSS-REFERENCE TO RELATED APPLICATIONS

The present application is a non-provisional application that claims the benefit of and priority to U.S. Provisional Application Ser. No. 62/585,373, the entire contents of which are incorporated herein by reference.

### FIELD

Embodiments of the present invention relate to the fields of physiological-signal processing, computational biology, health assessment and lifestyle quantification through wearable, nearable, ingestible and implantable sensors. In particular, embodiments of a platform for computing morbidity and mortality risk from digital health data to monitor risk and life style choices are presented.

### BACKGROUND

With recent advances in the quality of medication and healthcare, longevity of humans has increased significantly. However, this acquired longevity is not necessarily accompanied by acceptable health levels, but rather by multiple, and often complex, terminal health conditions. This co-occurrence of at least two chronic illnesses in an individual, termed multi-morbidity, with the prevalence amongst the elderly of any given nation usually exceeding 60%, does not only greatly diminish the quality of life for those affected, but has become one of the main challenges in, and burdens on, health-care and -insurance worldwide.

The monitoring of population health is essential not only in terms of global economy, but also as a measure of quality of life of a nation. Various measures of health have been developed and improved upon, which lies beyond the scope of this discussion.

In recent years, a growing body of research on how variation in physiological measurements may relate to long term outcomes, such as the risk of disease development and all-cause mortality, has emerged. In addition, a significant body of research exists that demonstrates the expected changes in physiological parameters in response to lifestyle choices such as performing aerobic exercise.

In survival analysis, several techniques are demonstrated in the scientific literature to quantify the health risk associated with various physiological variations. One of the more commonly used techniques, Cox regression, is often used to express the logarithm of all-cause mortality risk as a function of age (or time) plus a linear combination of demographic and physiological risk factors. By using the specific values of physiological parameters for an individual in such population based models, it is possible to compute mortality or morbidity risks and to compute an expected life expectancy in years.

Many studies, spanning across groups of up to millions of individuals, have been conducted to improve our understanding of the all-cause mortality and disease risk implications associated with variations in physiological parameter values. An example of such a parameter is the resting heart rate of a person, for which a dose dependent increase in the risk of all-cause mortality has been observed. The risk associated with such a physiological parameter is often expressed as the ratio of the risk that a person with said resting heart rate is exposed to, compared to the risk of an individual or population that holds a normative value for the parameter. This ratio of hazards (hazard for subject/hazard of individual or group with normative parameter value) is known as the hazard ratio and is available for many physiological parameters.

A unit, such as all-cause mortality risk, cause-specific risk, morbidity risk, and the hazard ratio or life expectancy, mediates the expression of the consequences of lifestyle choices as a single number with a common understandable unit, for example, years of life expectancy gained or lost associated with a specific choice, such as commencing an exercise program, or, taking up smoking. This enables the direct comparison of these choices and optimization of various lifestyle choices in a numerical fashion. Similarly, methods exist for converting the survival analysis associated with life expectancy figures to a so called biological age by calculating the equivalent age of a reference population for which the all-cause mortality risk is equal to the all-cause mortality risk of a specific individual in question, i.e., the risk equivalent age.

Research has also been performed to elucidate the positive correlation between changes in physiological parameters to lifestyle interventions exemplified by, but not limited to, diet and exercise regimes. An example of such a study is the response of overall aerobic fitness (as measured during a VO2max test) to exercise intervention. Depending on the frequency, duration, intensity and initial fitness levels of a participant, specific changes in VO2max have been observed. It is possible to capture such data in a model to extrapolate from past studies directly to the individual, whereby expected changes in VO2max value in response to a planned duration, intensity and frequency of exercise, may be provided. Similarly, it is possible to make predictions of planned weight loss values, based on estimates of an individual's metabolic rate and diet and how weight loss would affect VO2max value, which is expressed in ml/min/kg of body mass. Changes in blood pressure values in response to changes in dietary sodium levels is another area that has been well studied and where a projection of expected changes at the hand of a low sodium diet may be provided, given an online diary and/or blood pressure measurements.

Using body monitoring technology, it has also become possible to track the lifestyle changes implemented by users, in a passive way. By using a wrist worn wearable device equipped with one or more optical sensors as well as an accelerometer, the measuring of real-time heart rate and exercise activity is facilitated. Moreover, the passive tracking of the frequency, intensity and duration of exercise can be enabled which, in turn, could be used to compare measured behavior to planned behavior.

Physiological parameters such as body mass, blood pressure and VO2max may be tracked either now or in the foreseeable future, by using body monitoring technology. For example, various connected Wi-Fi scale models exist that automatically upload the weight of a user to a cloud server. Regarding wearable technology, some devices include a sub-maximal exertion protocol which may be employed in conjunction with an exercise treadmill to obtain frequent estimation of the VO2max value of said user. Similarly, a connected sphygmomanometer and/or less intrusive body monitoring technologies may be used to measure blood pressure values in a more continuous fashion, after which said values may be communicated to a cloud server. The platform proposed herein has the necessary architecture for considering data from such external services via API (Application Programming Interface) calls or other relevant methods for sharing and accessing data and calculations in an anonymized, Health Insurance Portability and Accountability Act (HIPAA) compliant manner.

### SUMMARY

Certain physiological parameters, exemplified by, but not limited to VO2max value, RHR, maximum heart rate and BMI are indicative of morbidity- and mortality risk. Embodiments of the claimed invention comprise methods by which data gathered from, for example, wearable devices, are used to track the value of these parameters to predict morbidity and mortality risk and derivatives thereof, such as life expectancy and biological age. In addition, lifestyle choices such as exercise can also be tracked to project how current lifestyle will affect said physiological parameters and also how that will affect morbidity- and mortality risk and derivatives thereof. Moreover, in the case of predicted life expectancy, the disclosure can produce a value in years, a single unit wherein the impact of different lifestyle choices can be expressed and compared against each other to make an optimal choice.

### BRIEF DESCRIPTION OF THE DRAWINGS/FIGURES

The accompanying drawings, which are incorporated herein and form a part of the specification, illustrate embodiments of the present disclosure and, together with the description, further serve to explain the principles of the disclosure and to enable a person skilled in the pertinent art to make and use the embodiments.

FIG. 1 illustrates VO2max aging trajectories at different activity levels with disks indicating endurance trained, squares indicating active, and diamonds indicating sedentary individuals. The arrows in the background indicate a vector field that may be used to project the ageing pattern for individuals of intermediate fitness levels, according to an embodiment.

FIG. 2 illustrates an example of VO2max projection over calendar year using a multivariate demographic model according to an embodiment, combined with a few interspersed measurements spanning from 2000-2016. In this case the individual has undergone substantial changes in body mass (varying from 78 Kg to 150 Kg), over this period. The solid blue line shows the historic data for the individual prior to 2017 and a projection of how VO2max will change after. Dashed lines indicate projections of future VO2max values from each point where physiological data was collected, resulting in a branched representation, according to an embodiment.

FIG. 3 illustrates depicts a survival curve for an individual undergoing drastic fluctuations in body mass and other physiological parameters, constructed by combining a background demographic based hazard rate with physiology specific hazard ratios derived from body monitoring data, according to an embodiment.

FIG. 4 illustrates projected VO2max gains, ascribed to different parts of an individual exercise session as shown inF the pie chart for exercise sections of different intensity if the exercise is assumed to be continued in this case at a frequency of 4 times per week, according to an embodiment.

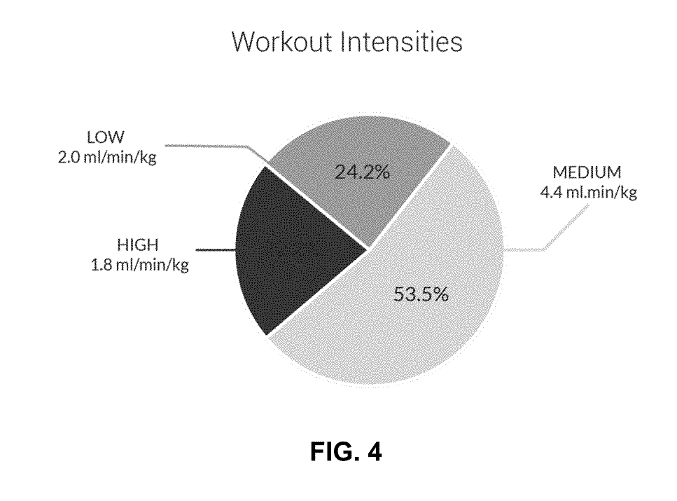

FIG. 5 illustrates data and processing flow for an example embodiment of the claimed invention with a lifestyle impact model projecting changes to physiological parameters and a health risk module determining health risk and derivatives such as life expectancy and biological age, according to an embodiment.

FIG. 6 is an example computer system useful for implementing various embodiments.

Embodiments of the present disclosure will be described with reference to the accompanying drawings. In the drawings, like reference numbers indicate identical or functionally similar modules.

### DETAILED DESCRIPTION

The exemplary embodiments described herein are provided for illustrative purposes, and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described below.

For purposes of this discussion, any reference to the term “module” shall be understood to include at least one of software, firmware, and hardware (such as one or more circuit, microchip, or device, or any combination thereof), and any combination thereof. In addition, it will be understood that each module may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

The following detailed description of the exemplary embodiments will so fully reveal the general nature of the disclosure that others can, by applying knowledge of those skilled in relevant art(s), readily modify and/or adapt for various applications such exemplary embodiments, without undue experimentation, without departing from the spirit and scope of the disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

Embodiments of the claimed invention improve upon the existing stratification process used by life insurers by offering a lower friction, more scalable, as well as a more physiologically sound and potentially dynamic stratification process underwritten by the translation from interventions pertaining to lifestyle (such as commencing an exercise program) to physiological parameter changes coupled to risk (such as VO2max value), to life- and/or health expectancy. Embodiments of the proposed platform implement not only planned life style interventions, but also past life style interventions, enabling the user to quantify how previous behavior translated to health gains.

For example, in an embodiment, the proposed platform is capable of translating past and planned lifestyle intervention such as exercise into physiological parameter changes, specifically in those physiological parameters coupled to morbidity and mortality risk, into dynamic life expectancy in the form of years of life gained or lost. Not only do changes in the physiological parameters mentioned translate into changes in life expectancy, but also into changes in health expectancy, as athletes that have a lifelong increase in VO2max compared to the general population, also experience an increased health-span that allows them to have youthful performance levels, even in advanced age. The physiological parameters targeted in embodiments and which have been tracked in large studies are typically those associated with both increased health and life expectancy such as increased fitness levels, optimal body mass index (BMI), lower resting heart rate (RHR) and optimal blood pressure.

Having several connected users, it also becomes possible to provide additional utility to users by allowing them to connect to each other and to share lifestyle interventions for which the success can be quantified based on accrued effects measured on the platform that connects these users. In this way, new diets, exercise programs, health supplements and many other lifestyle interventions can be tested and promoted, based on objectively measured evidence collected by the wearables/nearables/ingestible technologies connected to the proposed platform.

The disclosure is organized as follows. First, an example embodiment of an overall system with example inputs, outputs and different devices connected to it, is described. Second, example methods by which physiological measurements are translated into predictions of physiological risk are described. Third, an embodiment of the integration of risk to provide a life expectancy value is described. Finally, an example process of monitoring lifestyle behavior using the inputs to the platform, as well as prediction and presentation of physiological parameter changes and thereby health risk, via for example life expectancy changes, is described with respect to an embodiment.

An embodiment of the invention comprises a platform that gathers physiological data derived from connected sensors from individuals. Such a process will be referred to as body monitoring. Said data is then interpreted and/or transformed at the hand of models based on published research studies and/or privately funded studies, for example pilot projects with third parties having a vested interest in quantifying and improving health. In an embodiment, such an interpretation yields, for example and without limitation, one or more of the following: (i) risks of mortality, morbidity or disease development associated with the monitored person's physiology; (ii) life expectancy, biological age estimation and other forms of health quantification based on the monitored person's physiology; (iii) projected changes to physiological parameters based on lifestyle choices such as diet and exercise plans; (iv) projected modification to physiological parameters based on the measured behavior of the user; (v) actual modification to physiological parameters, by measuring said parameters via body monitoring data streams; (vi) modification of disease and all-cause mortality risk associated with life style plans, either as projected for a certain lifestyle plan, measured lifestyle intervention or measured changes to physiological parameters.

In an example survival analysis, an individual is considered to exist under a relatively low and relatively constant hazard of dying or developing disease at any given instant in time. Under such an assumption, there is no single deterministic life duration, but rather, a distribution of possible life expectancies with different probabilities implied. When the term life expectancy is used for an individual, it generally refers to the most likely duration of a lifetime. However, the assumption that the hazard is not completely constant is necessary, especially due to the known increasing risk of disease development and mortality with advanced age. In the early 1800s, Benjamin Gompertz demonstrated that human mortality risk increases exponentially with advancing age. A simple rule of thumb is that the risk of mortality doubles for every eight years of adult human life span.

The basic equation used to model survival is essentially an ordinary differential equation of the form:

$$\frac{dS}{dt} = - μ S ( t )$$

where S(t) represents the survival fraction (between 0 and 1) and μ is the mortality risk expressed as the fraction of survivors dying per time unit. In this way, even when considering a survival analysis for an individual, we are looking at the individual as if consisting of the average outcome of a population of clones, a fraction of which dies at every time step from birth to advanced age.

The mortality risk μ, is then a function of age, or t in the model, and typically follows an exponential doubling such that μ=μ₀e^{t/λ}, where lambda is proportional to the doubling time for risk, as mentioned before, being close to 8 years in human populations.

Studies have shown that the risk of mortality in a population also varies with physiological parameters, such as resting heart rate value. This implies that μ is not only a function of age, but also of the various physiological parameters that could be measured on an individual. Considering a parameter such as resting heart rate, it has been shown that there is a dose-dependent increase in mortality risk. In this case, the reported value is an increase in mortality risk of 16% for each 10 bpm increment. An individual with a 70 bpm resting heart rate would thereby display two 10 bmp increments over 50 bpm, resulting in a 1.16×1.16=1.3456-fold risk increase relative to a reference individual with a 50 bpm resting heart rate and where other factors are equal. Therefore, it is important to express the risk of mortality, μ, as a function not only of time, but also as a function of the physiological parameters of an individual, ρ, which would include factors that affect mortality risk, exemplified by, but not limited to resting heart rate, fitness level (VO2max), BMI, total sleep time, sleep quality, sleep time variability and blood pressure values. Symbolically this can be expressed by:

μ=μ₀e^{t/λ}e^{k(ρ-ρ}^{₀}⁾

where ρ represents said physiological parameter and ρ₀ represents a reference value for a parameter. We do not limit ourselves to the exponential form used here for illustrative purposes. For example, it is well known that the risk associated with BMI follows a bathtub like distribution, where very low or very high values are associated with increased risk.

In the preceding paragraph the hazard ratio associated with deviation in a physiological parameter, specifically heart rate, was treated as a constant value of 1.16 (16% risk increase) for every 10 bpm increase. Resting heart rate is an example of a physiological parameter that does not appear to change with age in an adult. However, this is not the case for many other physiological parameters, such as fitness level expressed as VO2max. To use a hazard ratio reported for VO2max, the age of the participant must be brought into account.

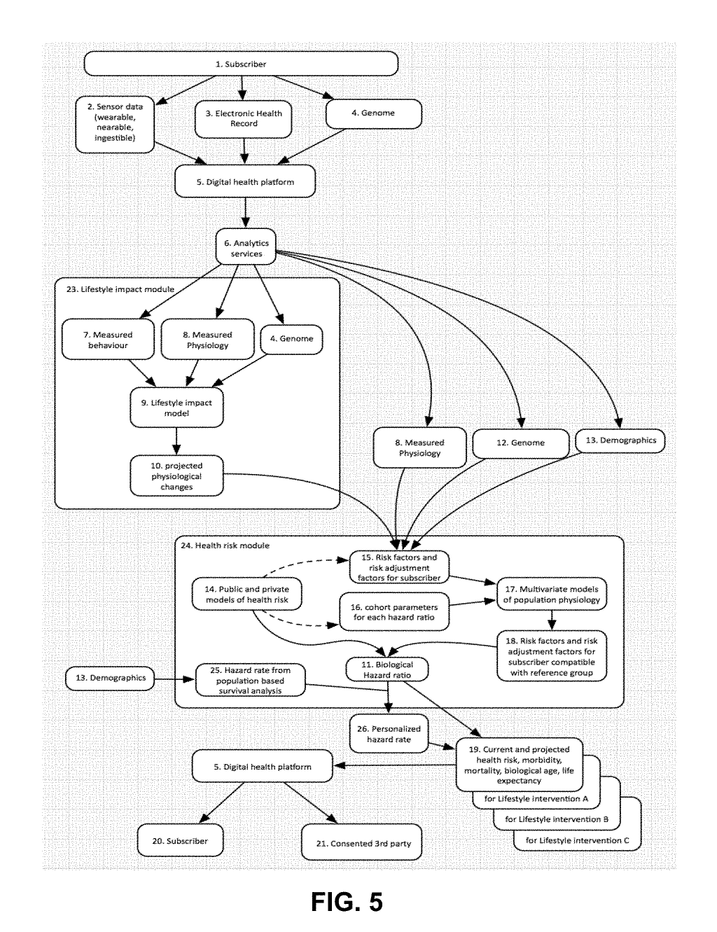

Referring to FIG. 5, in the case where the model used (14) does not explicitly adjust for age for evaluating risk and where the reference population (16) for that model does not match the participant in for example age, it is necessary to extrapolate the VO2max measured on the participant to an age that matches the reference population in terms of which this hazard is expressed (16, 17 and 18). For example, should the VO2max value of a 50-year-old be considered in such a case, while the average age of the reference group was 35, such a procedure would adjust his/her VO2max upward to the expected value at age 35.

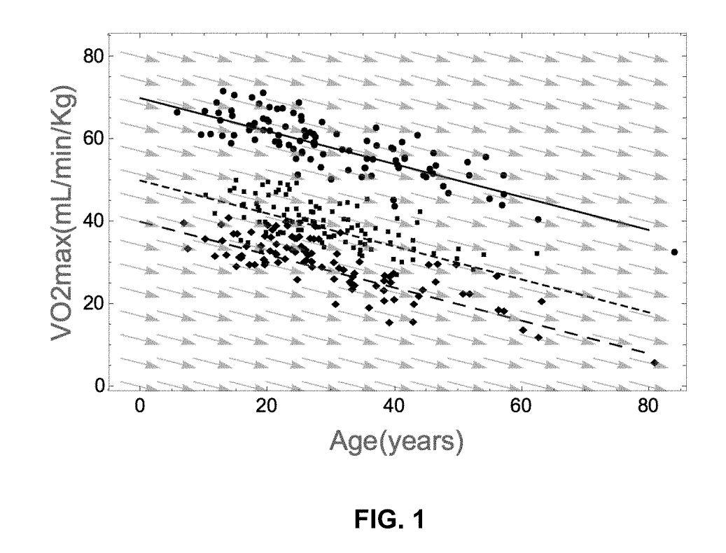

In the alternative case where the model used (14) does have regression coefficients for both age and VO2max, the age (13) and VO2max (11) of the participant in this case, 50 years, can simply be substituted into the risk model directly (14). Numerous studies providing demographic data that can be used to build such multivariate demographic models (17) exist. In FIG. 1 we show one such dataset upon which a model may be built to project a VO2max value for an individual forward or backward in time.

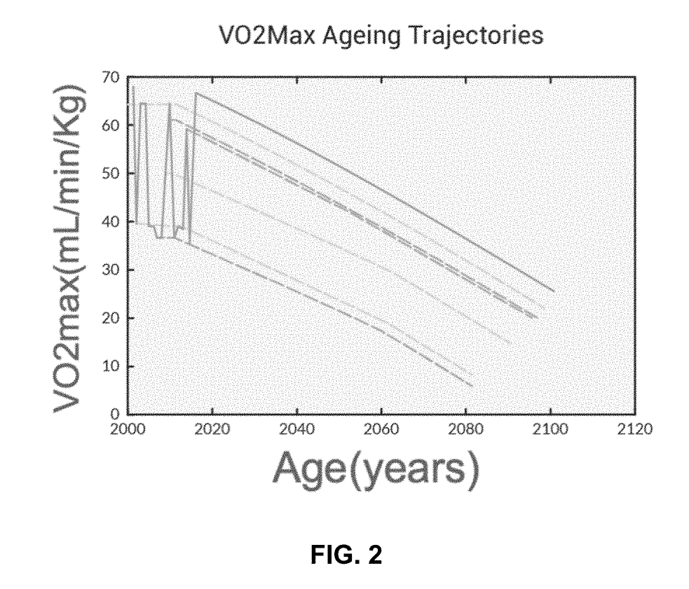

Factors such as exercise behavior and weight vary to create the different VO2max values over the course of a lifetime of an individual, as seen in FIG. 2. The hazard ratios (11) associated with the different physiological parameters (8) monitored via body monitoring technology may be combined, and will be addressed below. For varying physiological parameters, it then becomes possible to calculate a time varying hazard ratio μ for an individual, tied to his or her physiology. Given a background hazard rate for an individual from a large scale population survival analysis (14), which factors in demographic information exemplified by, but not limited to age, gender and nationality or address, it becomes possible to calculate a hazard rate (26) for an individual based on his/her physiological parameters and/or genetics (12) by multiplying the hazard ratios discussed earlier (11), with hazard rates derived from large scale population survival analysis (25) based on demographic information (13). In terms of an equation, this would be expressed as the personalized hazard rate (26) being the population hazard rate (25) multiplied by the combined individual hazard ratio (11).

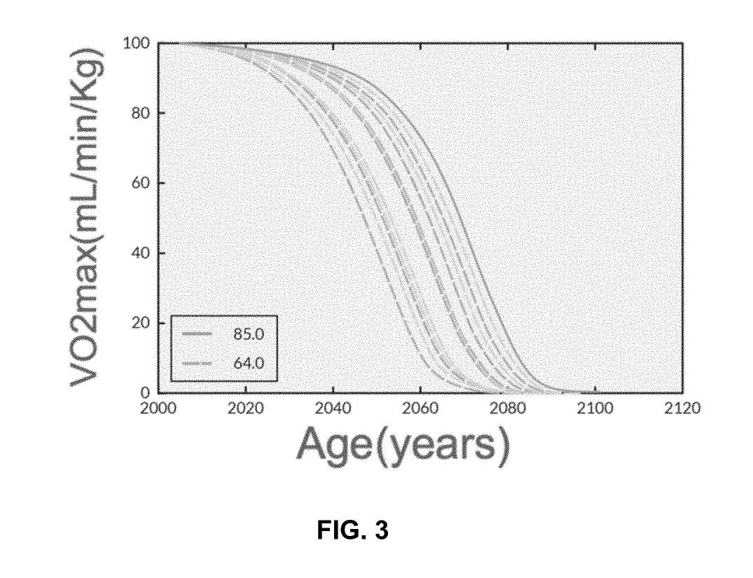

This personalized hazard rate (26) is the value required for μ, as described above. Considering the individual discussed in FIG. 2, we can calculate his physiological hazard ratio based on changes to VO2max and other physiological parameters (8), multiply it with an appropriate background hazard rate given demographics (13) such as gender, nationality and age to obtain a physiological hazard rate, where after the abovementioned ODE equation may be integrated, to arrive at both life expectancy values (64 years at maximum weight and 85 years at minimum weight), as well as an accompanying survival curve (FIG. 3) for the individual.

Less data exists to monitor the effect on mortality of focused lifestyle interventions such as exercise and diet programs, as most of the studies on risk associated with physiological parameters (14) did not involve coordinated interventions. However, for various reasons, the individuals enrolled in such studies do vary in the values of their physiology, lifestyle intervention being only one of many causative factors. Therefore, the understanding of how risk is affected when an individual changes physiological parameters through lifestyle intervention, is not fully understood. However, given the limited knowledge that we do have from population based risk studies, the most likely mortality risk that we would predict for the individual, should they partake in an all-cause mortality study, would be the risk associated with their current measurable physiological state. Considerable evidence has been presented on a narrow range of lifestyle interventions, notably smoking, where states such as abstaining, commencing or completely ceasing the behavior at different ages and in many individuals in a cohort, is prevalent. The literature on this hazard, that is understood to cause advanced aging and increased disease risk, shows that ceasing a habit such as smoking before age 40 leaves only 10% of the residual risk in later life, whereas ceasing at age 50 leaves around one third of the risk remaining throughout life. This substantiates to some degree the basic working assumption that the risk estimate associated with an individual's current physiological parameter values should shift in the direction of the physiological changes arrived at by lifestyle intervention. Further, at young ages, this assumption appears to hold more strongly, while there is some physiological damage that accrues when living in unhealthy states for extended periods of time, which permanently elevates risk compared to the general population.

The assumption that changes in physiological parameters are associated with changes in all-cause mortality risk or disease risk in a similar qualitative pattern as found in large cohorts, implies that the life expectancy of an individual, continuously changing in parameters such as fitness level (VO2max), resting heart rate, BMI or blood pressure, also changes continuously. Consequently, some estimate thereof may be calculated by using body monitoring data. Such information, when integrated over all tracked physiological parameter values and demographic parameters such as age, sex and nationality could then provide a time varying life expectancy that is subject to the variation in physiological parameters. Such an output may also be combined with another aspect of the technology—a planned lifestyle intervention such as weight loss, exercise programs or a low sodium diet may be used to, at the hand of the appropriate studies, predict changes to physiological parameters such as VO2max, BMI and blood pressure, and convert these projected changes to days of life expectancy gained or lost due to each proposed intervention (19). In this manner, it could also be possible to compare the benefits of not just alternate interventions, but also of multiple interventions in a single one-dimensional unit of years of life expectancy gained or lost. The advantage of such an approach to the subject using such a system (or consented third party) is that, based on an informed projection of their benefits, specific interventions may be selected. In addition, the lifestyle interventions (19) may be tracked at a behavioral level through body monitoring technology (2 and 7) and signal processing algorithms, as well as at a physiological parameter level by directly measuring weight, blood pressure, resting heart rate or VO2max values (2 and 3). Finally, factors such as genetics and their impact can be considered in the lifestyle impact model (9) to modify the predicted changes in for example VO2max in response to exercise, as some genetic variations influencing trainability are already known. Similarly, the subjective experience of exercise at a fixed intensity has also been shown to have a genetic component, and such information can be factored in to further personalize the lifestyle impact module (23).

In an embodiment used as part of a digital health platform (5), biological signals may be continuously detected and digitized from a range of supported devices into data streams and recorded by, for example, a wrist based device fitted with sensors (2). Consequently, data streams may be transformed into and represented as one or more physiological parameters (8) by analytics services (6), which may be internal or external to the digital health platform (5). These physiological parameters (8) can include, but are not limited to, VO2max value, heart rate, respiratory rate, genetics (12), blood pressure and sleep parameters such as total sleep time. Additionally, information requiring the execution of manual measurements (for example body mass, height and skinfold measurements) and/or verbal or written input from the individual (for example gender, exercise events and alcohol consumption), may be manually entered either onto the device itself and/or one or more external devices including, but not limited to smartphones, tablets or personal computers, and/or a cloud-based platform. Said data and/or information may be stored and/or processed and/or displayed on the device itself, and/or relayed wirelessly between the device and/or one or more of said external devices and/or a cloud-based platform.

Using the sensor data streams (2) that have entered the digital health platform (5), analytics services on or external to the digital health platform can be used to measure aspects of user behavior such as exercise and sleeping patterns (7). In an embodiment, a Lifestyle impact module (23) then considers the current physiology (8) of the subscriber (1) and optionally other context such as the subscriber's genomic data (12), to predict how physiological parameters will change over time. An example is a prediction of how a subscriber's (1) VO2max would change in response to the current frequency, intensity and duration of exercise given his/her current VO2max values and genetic propensity (12) to respond to exercise. This allows a projection of user physiology into the future which can optionally be included as input stream to the Health risk module (24).

In FIG. 3 is shown an example single exercise session by a user, that has been interpreted at the hand of a number of studies that have investigated the influence of exercise interventions expressed in terms of exercise intensity, frequency, duration and initial fitness level on VO2max. For a subject that makes use of body monitoring technology for which we have heart rate and activity data available via accelerometer and PPG technology, it is possible to quantify each of these factors (7) individually and, to estimate the VO2max gain (10) implied by a recorded exercise session, with some stated assumption about the frequency of this exercise pattern going forward, in the case of FIG. 3, performing this exercise 4 times per week.

By following a subject's behavior and physiological parameters in this way, it becomes possible to give feedback to the user in terms of a) the degree of planned adherence (expressed as intensity, frequency and duration of exercise in the specific example), b) the progress in measured physiological parameters (8) compared to projected changes in these parameters (10) and c) for example, life expectancy gains of a planned and executed lifestyle intervention (19).

Returning to FIG. 5, in an embodiment, the Health Risk module (24) considers measured physiology (8), demographics (6) and optionally the forward projected physiology of the user according to the Lifestyle impact model (23) and or the genomic profile (12) of the subscriber (1). Pooled together, these inputs are referred to as risk and risk-adjustment factors (15). Based on public and private models of health risk, such as Cox regression models from the literature, a combined model may be set up which operates on a set of health risk parameters (14). Depending on the complexity of the model and data available, various physiological parameters (8), including blood markers that form part of a subscriber's medical record (3) can be included, such as, but not limited to cholesterol and blood lipid profiles. Additionally, demographic (13) and genomic (12) factors can also be inputs to such a model. Based on the cohorts used to setup the parameters of the models used to construct 14, a set of cohort parameters (16) are also recorded that describe the groups used for expressing the hazard ratios. In cases where cohort parameters (16) have not been explicitly included as risk adjustment factors (15), a multivariate model of population physiology (17) can be used to transform a subscriber's physiology to a compatible physiology (18). This was exemplified in an earlier paragraph describing the case where the VO2max of a 50-year old was considered as risk adjustment parameter, but where the cohort was based on group with an age of approximately 35 years. In such a case, 17 functions as an ageing model that projects the physiology of the 50-year old to a higher value compatible with the younger cohort.

Once the set of risk and risk adjustment factors compatible with the cohort (18) has been calculated in 24, a biological hazard ratio (11) for the subscriber (1) can be calculated in combination with the health risk models (14). This hazard ratio reflects the risk for the individual due to biological factors described (including physiological and genomic) and to perform further analysis, hazard rates can be obtained from a larger population based model (25) that provides hazard rates at different ages or birth dates for a subscriber based on demographic information that is exemplified, but not limited to nationality. The product of this latter hazard rate and the biological hazard ratio (11) can be used to calculate a biological hazard rate (26) which can be used to calculate other derivatives for the subscriber (1) such as current and projected health risk, morbidity, mortality, biological age and life expectancy. Such information can also be delivered based on current physiology, projected physiology according to diet and or exercise or other life style intervention plans as indicated by the A, B and C in 19. In some of such cases, a single number such as life expectancy can then be used to determine and optimize a lifestyle intervention plan by choosing the intervention or combination of interventions that will yield the highest decrease in risk, lowest biological age or highest life expectancy value.

One consideration involves how hazards on different physiological parameters, reported in different studies and cohorts, may be combined to arrive at a holistic assessment of risk for an individual. A common practice in the literature on all-cause mortality or disease hazard is to correct for known, confounding variables to provide what is known as adjusted hazard ratios. In such cases, multiple physiological parameters can be fed into a Cox-regression model to get a hazard ratio for a subscriber. In cases where individual risk factors have separate studies outlining the risk for each, the interaction of these factors relating to risk is unknown and therefore one can reduce all covariation between two or more such parameters by statistical processes such as whitening of the multivariate distribution which guarantees that one only considers the non-correlated or orthogonal variation in each of the parameters. In such a case, the two or more hazard ratios derived from two or more studies can be combined by multiplying them due to their engineered independence.

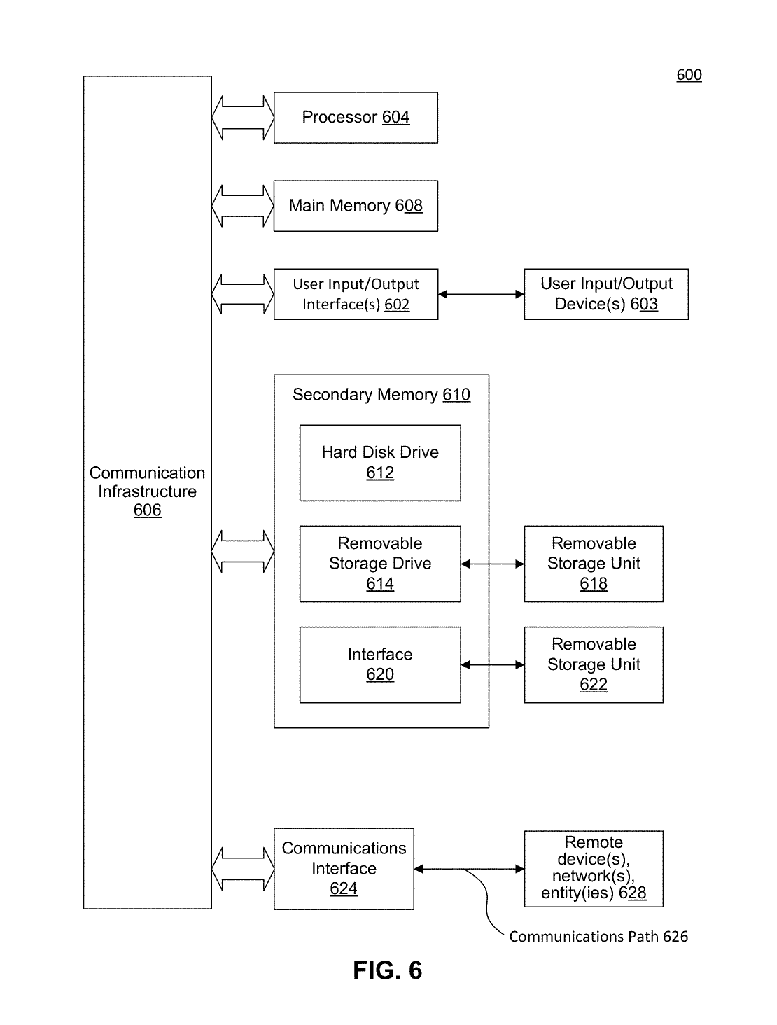

FIG. 6 is an example computer system that may be used to implement aspects of the systems illustrated in FIG. 5, or which may be specially programmed to implement aspects of the process discussed above. Computer system 600 includes one or more processors (also called central processing units, or CPUs), such as a processor 604. Processor 604 is connected to a communication infrastructure or bus 606.

One or more processors 604 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 600 also includes user input/output device(s) 603, such as monitors, keyboards, pointing devices, etc., that communicate with communication infrastructure 606 through user input/output interface(s) 602.

Computer system 600 also includes a main or primary memory 608, such as random access memory (RAM). Main memory 608 may include one or more levels of cache. Main memory 608 has stored therein control logic (i.e., computer software) and/or data.

Computer system 600 may also include one or more secondary storage devices or memory 610. Secondary memory 610 may include, for example, a hard disk drive 612 and/or a removable storage device or drive 614. Removable storage drive 614 may be a floppy disk drive, a magnetic tape drive, a compact disk drive, an optical storage device, tape backup device, and/or any other storage device/drive.

Removable storage drive 614 may interact with a removable storage unit 618. Removable storage unit 618 includes a computer usable or readable storage device having stored thereon computer software (control logic) and/or data. Removable storage unit 618 may be a floppy disk, magnetic tape, compact disk, DVD, optical storage disk, and/any other computer data storage device. Removable storage drive 614 reads from and/or writes to removable storage unit 618 in a well-known manner.

According to an example embodiment, secondary memory 610 may include other means, instrumentalities or other approaches for allowing computer programs and/or other instructions and/or data to be accessed by computer system 600. Such means, instrumentalities or other approaches may include, for example, a removable storage unit 622 and an interface 620. Examples of the removable storage unit 622 and the interface 620 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 600 may further include a communication or network interface 624. Communication interface 624 enables computer system 600 to communicate and interact with any combination of remote devices, remote networks, remote entities, etc. (individually and collectively referenced by reference number 628). For example, communication interface 624 may allow computer system 600 to communicate with remote devices 628 over communications path 626, which may be wired and/or wireless, and which may include any combination of LANs, WANs, the Internet, etc. Control logic and/or data may be transmitted to and from computer system 600 via communication path 626.

In an embodiment, a tangible apparatus or article of manufacture comprising a tangible computer useable or readable medium having control logic (software) stored thereon is also referred to herein as a computer program product or program storage device. This includes, but is not limited to, computer system 600, main memory 608, secondary memory 610, and removable storage units 618 and 622, as well as tangible articles of manufacture embodying any combination of the foregoing. Such control logic, when executed by one or more data processing devices (such as computer system 600), causes such data processing devices to operate as described herein.

### Example Embodiments

Example embodiments of the present invention may include the following features, alone or in combination where applicable:

A1. A system, comprising a first plurality of sensors configured to produce their respective plurality of signals related to a physiology of a first individual; and a first device configured to receive the first plurality of signals from the sensor and to generate a first plurality of data streams based on the first plurality of signals; and a second device different from the first device and configured to receive at least one of the data stream and a derivative of the data steam from the first device and to transmit the at least one of the data steam and the derivative of the data stream to a first cloud computing platform (also referred to herein as a cloud computing device), wherein at least one of the first device and the second device and the first cloud computing platform is configured to derive a first plurality of physiological parameters from the data stream, wherein the first cloud based platform is configured to receive the first plurality of physiological parameters from the second device; derive one or more morbidity or mortality associated parameters from the first plurality of physiological parameters; and transmit the mortality or morbidity related parameters to computing devices of any of the first individual and a first permitted third party via the cloud based platform.

A2. The system of embodiment A1, wherein the morbidity or mortality associated parameters are replaced by the values of the first physiological parameters in the context of respective ranges associated with increasing/decreasing levels of mortality/morbidity.

A3. The system of embodiment A1, wherein the permitted third party is an insurance provider and the individual is a subscriber to the insurance provider, and wherein the first mortality or morbidity associated parameter is used to determine at least one of a premium, a benefit, and a reward for the individual.

A4. The system of embodiment A1, where at least one of the transmission of the plurality of data streams or plurality of derivatives of the data streams to the first cloud computing platform is handled by at least one of the second device or the first device.

A5. The system of embodiment A4, wherein the at least one of the first device and the second device and the first cloud platform is further configured to derive a plurality of behavioral parameters from the data stream, wherein at least one of the morbidity related parameters and the mortality related parameters of the first individual is expressed as at least one of:
(i) the plurality of physiological parameters or the plurality of behavioral parameters relative to a reference value of at least one of the plurality of physiological parameters or the plurality of behavioral parameters, the reference value for a particular physiological parameter or particular behavioral parameter being associated with a reference mortality or morbidity risk level for the particular physiological parameter or particular behavioral parameter,
(ii) a mortality or morbidity hazard ratio with respect to a behavioral parameter or a physiological parameter,
(iii) a relative risk with respect to a behavioral parameter or a physiological parameter, or
(iv) a score expressing an ordering of an increasing or decreasing level of mortality or morbidity risk.

A6. The system of embodiment A5, wherein the at least one of the first device and the second device and the first cloud platform is further configured to use the at least one of a plurality of first physiological parameters and first behavioral parameters to estimate a combined mortality associated parameter or combined morbidity associated parameter by using a model, exemplified by, but not limited to Cox regression, wherein at least one of the following holds:
(a) at least two of said physiological or behavioral parameters were used to build the model, thereby de-confounding the physiological and behavioral parameters entered into the model to predict a combined mortality or morbidity associated parameter. or
(b) de-correlating at least one of a first subset of the first physiological parameters or behavioral parameters with respect to a second subset and calculating at least one of the combined morbidity and combined mortality associated parameters by the procedure of using separate models or algorithms for calculating mortality or morbidity associated parameters which have been trained or built on said first and second subset and using multiplication or similar applicable operation to combine the mortality or morbidity associated parameters produced by said separate models or algorithms into said single combined mortality or morbidity associated parameter.

A7. The system of embodiment A6, wherein the first individual and a second individual are each part of a social network having a lifestyle plan, and wherein at least one of the combined morbidity and combined mortality associated parameter and plurality of physiological parameters and behavioral parameters is used to evaluate progress on the lifestyle plan or to project the benefit of a change to the lifestyle plan or to compute an alternative lifestyle plan.

A8. The system of embodiment A6, wherein the at least one of the first device and the second device and the cloud platform is further configured to adjust a parameter of an algorithm for computing life expectancy of the first individual by adjusting the overall risk modification value of the first individual and to determine a life expectancy value for the individual based on the adjusted parameter, wherein the parameter determines the estimated chance of the first individual surviving to the life expectancy value, and wherein the algorithm for computing the estimated survival of the first individual uses the morbidity risk to quantify a chance of disability of the first individual.

A9. The system of embodiment A6, wherein the at least one of the first device and the second device and the first cloud platform is further configured to determine a second risk modification factor for a particular age of a second individual different than the first individual, wherein the age of the second individual is different than the age of the first individual, wherein the second risk modification factor is based on an aging algorithm to predict one or more age related changes for the plurality of physiological parameters.

A10. The system of embodiment A6, wherein the at least one of the first device and the second device and the first cloud platform is further configured to determine a second risk modification factor based on genetic data of the individual.

A11. The system of embodiment A6 wherein the at least one of the first device and the second device and the first cloud platform is further configured to calculate an estimate of the benefit of behavioral choices as recorded and calculated from the plurality of sensor data-streams for the individual based on the combined mortality or morbidity associated parameter.

A12. The system of embodiment A6, wherein the at least one of the first device and the second device and the first cloud platform is further configured to calculate a projection of at least one of a first risk and a first benefit associated with a range of different planned lifestyle choices for the individual based on at least one of the combined morbidity and mortality parameter.

A13. The system of embodiment A5, wherein at least one of the mortality and or morbidity associated parameters are scores established in the scientific literature, exemplified by, but not limited to the Framingham Risk score associated with cardiovascular risk morbidity.

A14. The system of embodiment A5, wherein at least one of the morbidity or mortality associated parameters are expressed as a biological age, indicating the age of a reference group for which the morbidity or mortality associated parameter would attain approximately the same value.

A15. The system of embodiment A5, where the system receives from a consented third party, data from which behavioral or physiological or medical parameters can be derived and uses these as additional sources for determining at least one of a plurality of mortality and morbidity associated parameters and combined mortality and morbidity associated parameters.

Based on the teachings contained in this disclosure, it will be apparent to persons skilled in the relevant art(s) how to make and use embodiments of the present disclosure using data processing devices, computer systems and/or computer architectures other than that shown in FIG. 6. In particular, embodiments may operate with software, hardware, and/or operating system implementations other than those described herein.

What is claimed is:

1. A system, comprising:
a plurality of sensors configured to produce a plurality of signals related to a physiology of a first individual;
a first device configured to receive the plurality of signals from the plurality of sensors and to generate a plurality of data streams based on the plurality of signals; and
a second device different from the first device and configured to receive the plurality of data streams, wherein the second device is configured to be in communication with a cloud computing device,
wherein at least one of the first device, the second device, or the cloud computing device is further configured to:
derive a derivative of each of the plurality of data streams,
derive a plurality of physiological parameters from the plurality of data streams,
derive one or more morbidity or mortality associated parameters based on the plurality of physiological parameters, and
transmit the morbidity or mortality associated parameters to one or more computing devices of the individual or a permitted third party.

2. The system of claim 1, wherein the morbidity or mortality associated parameters are replaced with the plurality of physiological parameters in the context of ranges associated with increasing or decreasing levels of mortality.

3. The system of claim 1, wherein the permitted third party is an insurance provider and the individual is a subscriber to the insurance provider, and wherein the morbidity or mortality associated parameters are used to determine at least one of a premium, a benefit, and a reward for the individual.

4. The system of claim 1, wherein at least one of the first device and the second device is configured to:
derive the derivative of each of the plurality of data streams; and
transmit at least one of the plurality of data streams and the plurality of derivatives of the data streams to the cloud computing device.

5. The system of claim 4, wherein at least one of the first device, the second device, or the cloud computing device is further configured to:
derive a plurality of behavioral parameters from the plurality of data streams,
wherein at least one of the morbidity associated parameters and the mortality associated parameters of the first individual is expressed as at least one of:
(i) the plurality of physiological parameters or the plurality of behavioral parameters relative to a reference value of at least one of the plurality of physiological parameters or the plurality of behavioral parameters, the reference value for a particular physiological parameter or particular behavioral parameter being associated with a reference risk level for the particular physiological parameter or particular behavioral parameter,
(ii) a mortality or morbidity hazard ratio with respect to a behavioral parameter or a physiological parameter,
(iii) a relative risk with respect to a behavioral parameter or a physiological parameter, or
(iv) a score expressing an ordering of an increasing or decreasing level of a mortality or morbidity risk.

6. The system of claim 5, wherein at least one of the first device, the second device, or the cloud computing device is further configured to:
determine an estimated combined mortality associated parameter or an estimated combined morbidity associated parameter based on at least one of the plurality of physiological parameters and the plurality of behavioral parameters,
wherein the estimated combined mortality associated parameter or combined morbidity associated parameter is determined using at least one of a first model and a second model,
wherein the first model incorporates and de-confounds at least two of the plurality of physiological parameters or the plurality of behavioral parameters, and
wherein the second model incorporates and de-correlates at least a first one of a first subset of the plurality of physiological parameters or plurality of behavioral parameters with respect to a second one of a second subset of the plurality of physiological parameters or the plurality of behavioral parameters;
determine the morbidity or mortality associated parameters based on different models, wherein the different models are based on the first and second subsets of the plurality of physiological parameters or the plurality of behavioral parameters; and
combine the mortality or morbidity associated parameters produced by said separate models into the combined mortality parameter or the combined morbidity associated parameter.

7. The system of claim 6, wherein the first individual and a second individual are each part of a social network having a lifestyle plan, and wherein at least one of the combined morbidity associated parameter, the combined mortality associated parameter, and the plurality of physiological parameters and behavioral parameters is used to evaluate progress on the lifestyle plan, to project the benefit of a change to the lifestyle plan, or to compute an alternative lifestyle plan.

8. The system of claim 5, wherein the at least one of the first device and the second device is further configured to:
adjust a parameter of a model for computing life expectancy of the first individual by adjusting the overall risk modification value of the first individual, and
determine a life expectancy value for the individual based on the adjusted parameter,
wherein the parameter determines an estimated chance of the first individual surviving to the life expectancy value using the morbidity risk to quantify a chance of disability of the first individual.

9. The system of claim 6, wherein at least one of the first device, the second device, and the cloud computing device is further configured to:
determine a second risk modification factor for a particular age of a second individual different from the first individual,
wherein the age of the second individual is different from the age of the first individual, and
wherein the second risk modification factor is based on an aging model to predict one or more age related changes of the plurality of physiological parameters.

10. The system of claim 6, wherein at least one of the first device, the second device, and the cloud computing platform is further configured to determine a second risk modification factor based on genetic data of the individual.

11. The system of claim 6, wherein at least one of the first device, the second device, and the cloud computing platform is further configured to:
calculate an estimated benefit of a plurality of behavioral choices for the first individual from the plurality of data streams based on at least one of the combined morbidity associated parameter or the combined mortality associated parameter.

12. The system of claim 5, wherein at least one of the first device, the second device, and the cloud computing platform is further configured to:
calculate a projection of at least one of a first risk and a first benefit associated with a range of different planned lifestyle choices for the individual based on at least one of the combined morbidity associated parameter and the combined mortality associated parameter.

13. The system of claim 5, wherein the at least one of the morbidity associated parameter or mortality associated parameter provides a score for the individual.

14. The system of claim 5, wherein the at least one of the morbidity associated parameter or the mortality associated parameter is expressed as a biological age associated with a reference group for which the at least one of the morbidity associated parameter and mortality associated parameter would attain the same value.

15. The system of claim 5, wherein at least one of the first device, the second device, and the cloud computing platform is further configured to:
receive data from a consented third party for deriving at least one of the plurality of behavioral parameters, the plurality of physiological parameters, and a plurality of medical parameters; and
using the received data to determine at least one of the plurality of mortality associated parameters, morbidity associated parameters, a combined mortality associated parameter, and a combined morbidity associated parameter.
